(19) United States
(12) Patent Application Publication
(10) Pub. No.: US 2016/0022473 A1
(43) Pub. Date: Jan. 28, 2016

(54) INTRA-ORAL DEVICE FOR MANIPULATING THE MUSCULUS UVULAE AND UVULA

(71) Applicant: Michael Winters, Putnam Valley, NY (US)

(72) Inventor: Michael Winters, Putnam Valley, NY (US)

(21) Appl. No.: 14/807,367

(22) Filed: Jul. 23, 2015

Related U.S. Application Data
(60) Provisional application No. 62/027,970, filed on Jul. 23, 2014.

(51) Int. Cl. A61F 5/56

(52) U.S. Cl. CPC A61F 5/566

(57) ABSTRACT

An intra-oral device for manipulating the musculus uvulae and uvula is provided comprised of a support member attached to a dental splint having a means to regulate the musculus uvulae and uvula by use of an adjustment bend and about parallel and rear configuration, a method manipulating the tissue of the musculus uvulae and the uvula to bring about relief for snoring, sleep apnea and other similar or related disorders to improve a patient's health by maintaining unblocked air passage from the oropharynx to the nasopharynx.

### RELATED APPLICATION

This application claims the benefit of U.S. (Provisional) Application No. 62/027,970, filed Jul. 23, 2014, which is hereby incorporated herein by reference.

### BACKGROUND OF THE INVENTION

The present application relates to intra-oral devices, in particular to intra-oral devices used to reduce or eliminate snoring, sleep apnea and other similar or related disorders.

Just about everyone snores occasionally. If snoring happens frequently it can affect the quantity and quality of a person's sleep, and that of family members and roommates. Persons who snore often have floppy tissue that is more prone to vibrate. That tissue can be in throat, nasal cavity and structure of the soft palate, inclusive of the musculus uvulae which moves the uvula.

The most common form of sleep apnea is obstructive sleep apnea. With this condition, the airway collapses or becomes blocked during sleep for much the same reasons that produce snoring and cause shallow breathing or breathing pauses.

The similar or related disorders connected to snoring and sleep apnea makes up a substantially long list. This emphasizes the fact that no treatment or product has approached a full solution to this multitude of disease problem states. As an example some related disorders are Congestive Heart Failure, Hypothyroidism, High Blood Pressure, Anxiety and Gastroesophageal Reflux Disease to name a few.

More specifically, contributing tissue problems are prone to exist in the middle pharyngeal and nasopharyngeal regions of the head. During sleep air passage between the soft palate and pharyngeal wall can become blocked for at least ten seconds giving rise to the problem.

Various health initiatives, surgery and devices, such as Positive Airway Pressure and Mandibular Advancement Splints, are currently implemented as treatments. For some individuals these treatments can be effective and for others give only marginal relief.

Attempts have been made to manipulate the entire velum as a solution to snoring, sleep apnea and other similar or related disorders. Broad in attempt, this kind of a solution lacks the specificity required to often times resolve issues maintaining air passage from the oropharynx to the nasopharynx.

Accordingly there is a need for a device and method that directly manipulates, more specifically, the tissue of the musculus uvulae and the uvula to bring about relief for snoring, sleep apnea and other similar or related disorders, in an efficient, safe and practical way, to improve a patient's health by maintaining unblocked air passage from the oropharynx to the nasopharynx.

### SUMMARY OF THE INVENTION

An intra-oral device is provided that includes a support member coupled to a dental splint, the support member including a first structure extending outward from the dental split, a second structure extending outward from the first structure and upward toward a roof of a user's mouth, a third structure extending from the second structure toward a rear of the user's mouth, the third structure terminating in an upwardly curved configuration, and a sling attached to the third structure.

In at least one embodiment, the first structure is shaped as a loop.

In at least one embodiment, the first structure is shaped so that the loop lays in a plane.

In at least one embodiment, the second structure comprises at least one adjustable bend sufficiently malleable to adjust the support member for proper support in a user's mouth.

In at least one embodiment, the second structure comprises a pair of adjustable bends.

In at least one embodiment, the third structure comprises a pair of rods terminating in an upwardly curved V-shape.

In at least one embodiment, the sling structure forms a concave shape in the V-shaped configuration.

In at least one embodiment, the third structure follows a contour of the roof of a user's mouth.

In at least one embodiment, the curved configuration is shaped to straddle an end of the user's soft pallet.

In at least one embodiment, the sling is shaped to support a user's uvula.

In at least one embodiment, the sling is shaped to limit movement of a user's uvula.

In at least one embodiment, the sling is made of a flexible material.

In at least one embodiment, the support member is made of at least one of: a rod, a tube, and a wire.

Additional aspects of the present invention will be apparent in view of the description which follows. An intra-oral device for manipulating the musculus uvulae and uvula as described herewith.

### BRIEF DESCRIPTION OF THE FIGURES

FIG. 1 is a perspective view of the right side and the back of the intraoral device according to one embodiment;

FIG. 2 is a left side view thereof, the right side being similar;

FIG. 3 is a top view thereof;

FIG. 4 is a bottom view thereof;

FIG. 5 is a front view thereof; and

FIG. 6 is a back view thereof

FIG. 7 is a section view of the invention in use.

FIG. 8 is a section view of another embodiment of the invention in use.

### DETAILED DESCRIPTION OF THE INVENTION

Subject matter will now be described more fully hereinafter with reference to the accompanying drawings, which form a part hereof, and which show, by way of illustration, exemplary embodiments in which the invention may be practiced. Subject matter may, however, be embodied in a variety of different forms and, therefore, covered or claimed subject matter is intended to be construed as not being limited to any example embodiments set forth herein; example embodiments are provided merely to be illustrative. It is to be understood that other embodiments may be utilized and structural changes may be made without departing from the scope of the present invention. Likewise, a reasonably broad scope for claimed or covered subject matter is intended. The following detailed description is, therefore, not intended to be taken in a limiting sense.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of exemplary embodiments in whole or in part.

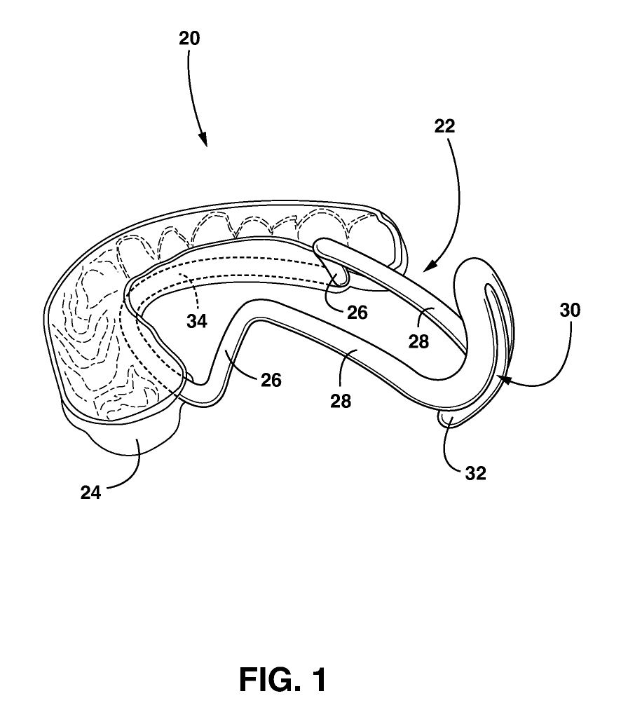

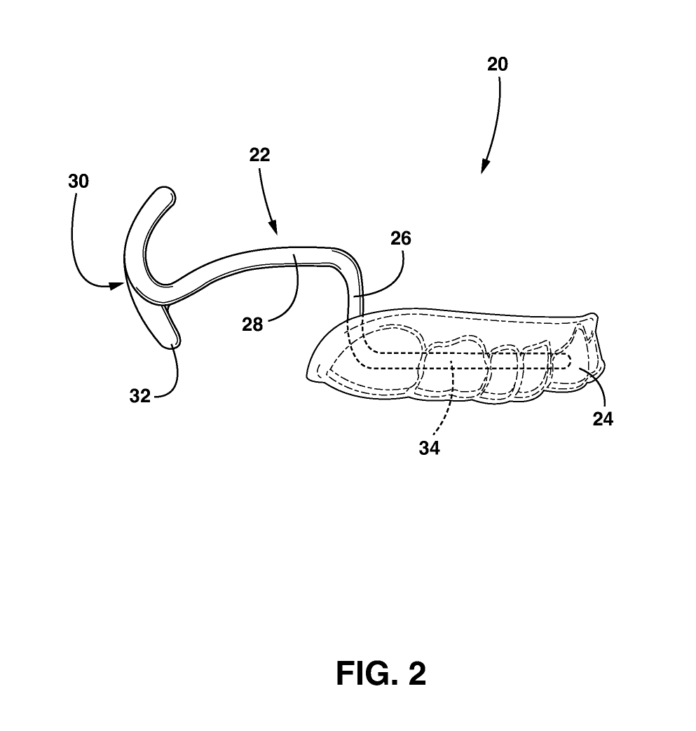

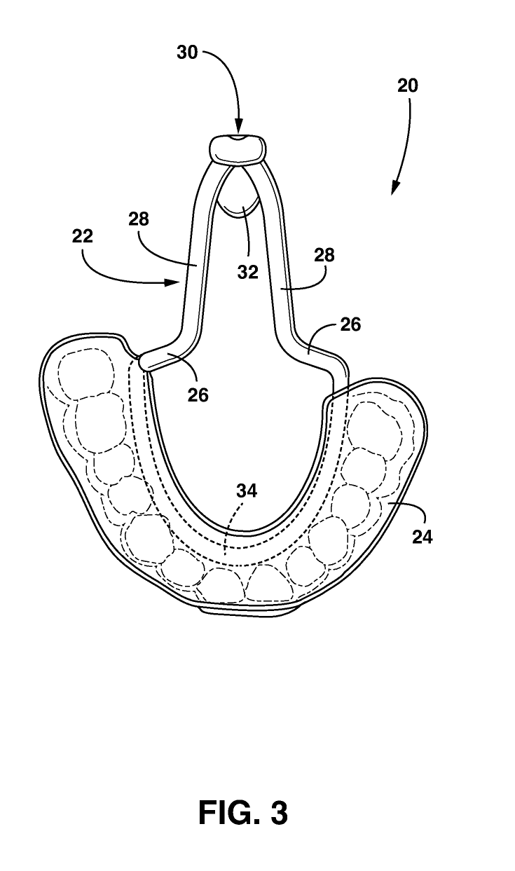

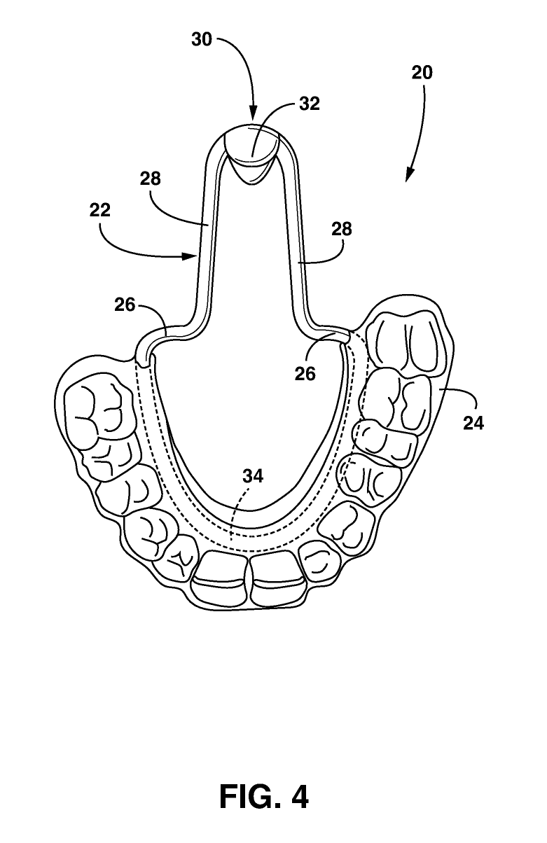

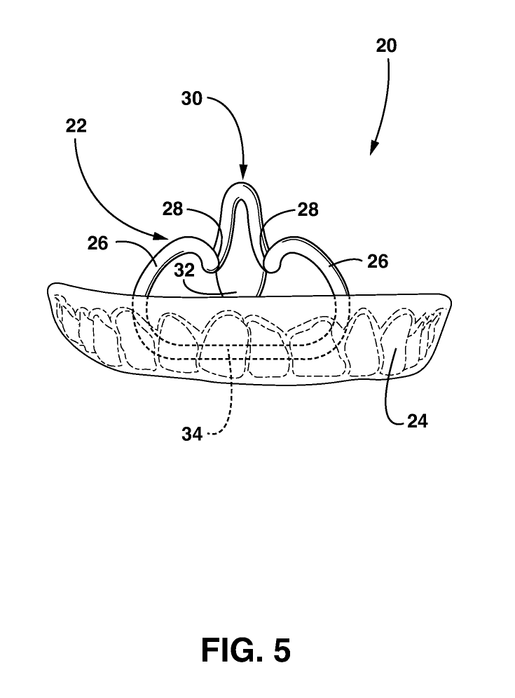

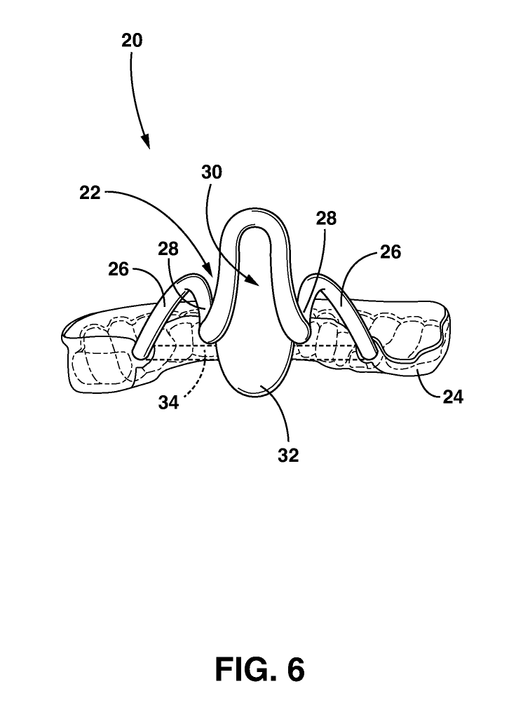

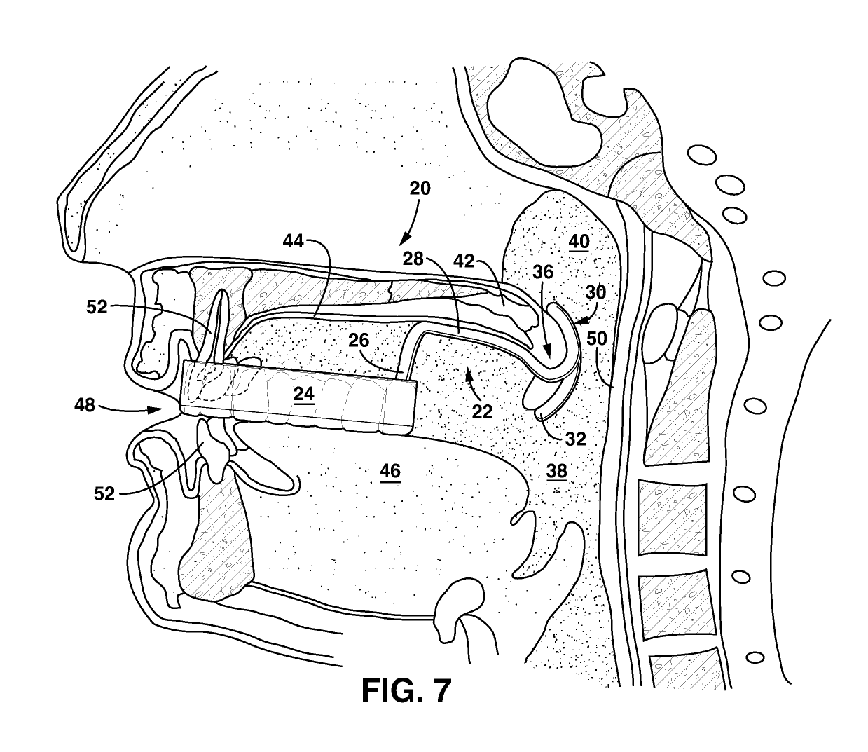

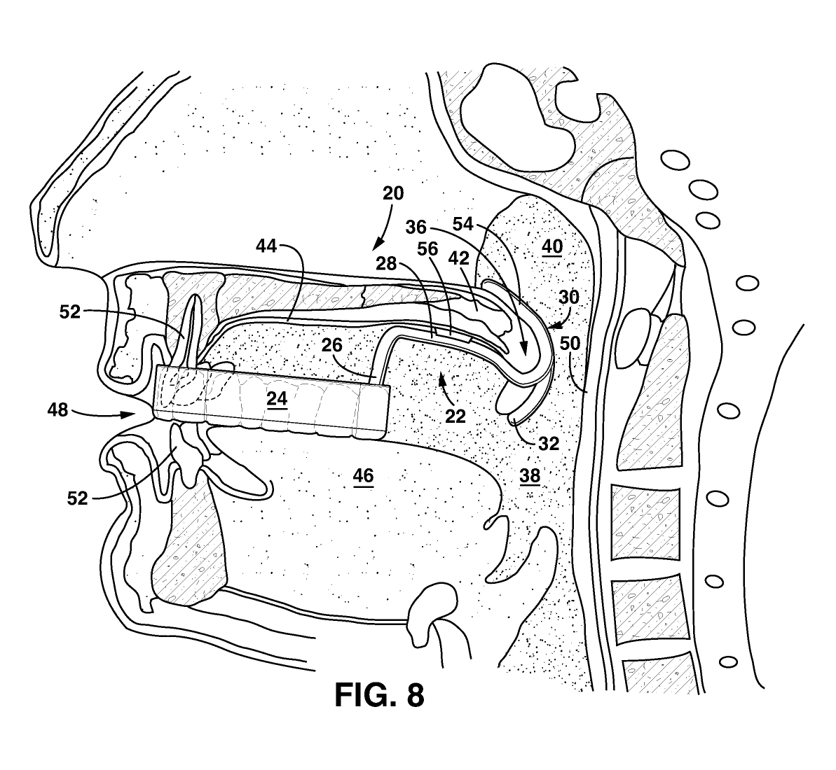

Referring to FIG. 1 through FIG. 8, various views of the intra-oral device for manipulating the musculus uvulae and uvula 20 according to at least one embodiment of the present device disclosed herein is shown. An intra-oral device for manipulating the musculus uvulae and uvula 20 reduces or eliminates snoring, and obstructive sleep apnea in the middle pharyngeal 50 and nasopharyngeal 40 regions, specifically regarding the blockage of air-passage between the soft palate 42 and pharyngeal wall 50 during sleep by manipulation of the uvula and uvulae musculus 36 to maintain air-passage from the oropharynx 38 to the nasopharynx 40 and vice versa.

An intra-oral device for manipulating the musculus uvulae and uvula 20 consists of a support member 22, which is preferably a non-corrosive, relatively rigid rod or tubular structure, which is stabilized by a dental splint 24 fixed to or otherwise part of the support member 22. The dental splint 24 may be a thermoform material laminated to a first loop 34 of the support member 22 so that loop 34 may be positioned continuously along the inside of the maxillary teeth 52. The structure of the loop 34 is preferably formed to sit in a single plane. The support member may include second structures, adjustment bends 26 extending from each end of the loop 24, in an upward direction toward the roof of the user's mouth 48. The second structures 26 may be essentially orthogonal to the plane that the first loop 34 lays. The second structures 26 continue toward the rear of the user's throat in third structures 28, in an essentially parallel configuration, that follows the contour of the roof 44 of the user's mouth 48. The third structures 28 continue rearward to terminate in an upwardly curved configuration 30, forming a V-shape that fits into the user's nasopharynx 40. The concave sling structure 32 extends from the inside/underside of the V-shape configuration, and is preferably made of somewhat flexible material, durometer scale of about Shore 60A. The sling 32 may be attached to the underside of rear configuration 30 by injection molding, or other means, and preferably extending downward to support the length of the uvula 36, thereby inhibiting the uvula 36 from moving or otherwise approaching the oropharynx 38. The structure at adjustment bend 26 permits is sufficiently malleable to permit adjustment of the support member 22 for properly locating the member 22 against the uvula and uvulae musculus 36, front to rear, creating variable separation of soft palate 42 from the pharyngeal wall 50 as required for each individual patient.

A dental splint 24 may be a two-ply construction and the inner ply, is a flexible material about durometer of Shore 40A, and can be easily removed with fingers from the top of dental splint 24. The outer ply is a more ridged material, about durometer of Shore 95A, and permanently encases loop 34 in its bottom structure.

An intra-oral device for manipulating the musculus uvulae and uvula 20 can be made from a rod, tube, wire, coated wire or other linear material known in the arts. Materials such as plastic, metal and composite materials can be used to create a member of about 0.12 inches in diameter forming a support member 22, a loop 34 and a rear configuration 30. A support member 22 has an about parallel configuration 28 that measures about 0.70 inches across and about 1.3 inches in length. A loop measures about 1.5 inches across by about 1.5 inches in length. A rear configuration 30 measures about 0.35 inches across and about 1.07 inches in length. A concave sling 32 measures about 0.4 inches across and protrudes about 0.3 inches below the bottom of about parallel configuration 28 in a somewhat oval or triangular shape. These dimensions are exemplary of one embodiment and can have a proportional variance with a patient's anatomy.

The shape of rear configuration 30 though over-all size may vary depending on patient's anatomy provides maximum comfort during swallowing and minimal gag-reflex during installation and use of an intra-oral device for manipulating the musculus uvulae and uvula 36.

Another embodiment of an intra-oral device for manipulating the musculus uvulae and uvula 20 may include; a.) an extension 54 or extensions 54 of rear configuration 30 that dampens vibration of the tissues in the upper nasopharynx 40. b.) A suction apparatus 56 may be secured to about parallel configuration 28 to dampen vibrations of the upper soft palate 42. An extension 54 can measure about 0.3 inches in length or other suitable dimension and is made of like materials previously described. A suction apparatus 56 can be cup like in shape or any suitable shape or configuration to further dampen vibrations by securing an intra-oral device for manipulating the musculus uvulae and uvula 20 to the upper soft palate 42.

The present invention is described further in the following Example, which is set forth to aid in the understanding of the invention, and should not be construed to limit in any way the scope of the invention as defined in the claims which follow thereafter.

### EXAMPLE

An intra-oral device for manipulating the musculus uvulae and uvula 20 is individually fitted by the dental technician or medical professional, during which time a patient's dental impressions and anatomical measurements are taken. During this impression and measurement process, a dental technician is mindful of fitting the narrow termination of rear configuration 30, appropriately capturing uvula and uvulae musculus 36, by regulation of adjustment bend 26 moving about parallel configuration 28 in a lateral, horizontal direction from the front to the back of the mouth 48 to acquire correct manipulation of the musculus uvulae and uvula 36, with the objective of reducing or eliminating snoring and obstructive sleep apnea in the middle pharyngeal 50 and nasopharyngeal 40 regions, specifically regarding the blockage of air-passage between the soft palate 42 and pharyngeal wall 50 during sleep to maintain air-passage from the oropharynx 38 to the nasopharynx 40 and vice versa.

FIG. 1 through FIG. 8 are conceptual illustrations allowing for an explanation of the present invention. Notably, the figures and examples above are not meant to limit the scope of the present invention to a single embodiment, as other embodiments are possible by way of interchange of some or all of the described or illustrated elements. Moreover, where certain elements of the present invention can be partially or fully implemented using known components, only those portions of such known components that are necessary for an understanding of the present invention are described, and detailed descriptions of other portions of such known components are omitted so as not to obscure the invention. In the present specification, an embodiment showing a singular component should not necessarily be limited to other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present invention encompasses present and future known equivalents to the known components referred to herein by way of illustration.

The foregoing description of the specific embodiments will so fully reveal the general nature of the invention that others can, by applying knowledge within the skill of the relevant art(s) (including the contents of the documents cited and incorporated by reference herein), readily modify and/or adapt for various applications such specific embodiments, without undue experimentation, without departing from the general concept of the present invention. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

While various embodiments of the present invention have been described above, it should be understood that they have been presented by way of example, and not limitation. It would be apparent to one skilled in the relevant art(s) that various changes in form and detail could be made therein without departing from the spirit and scope of the invention. Thus, the present invention should not be limited by any of the above-described exemplary embodiments, but should be defined only in accordance with the following claims and their equivalents.

What is claimed is:

1. An intra-oral device comprising a support member coupled to a dental splint, the support member comprising:
a first structure extending outward from the dental split,
a second structure extending outward from the first structure and upward toward a roof of a user's mouth,
a third structure extending from the second structure toward a rear of the user's mouth, the third structure terminating in an upwardly curved configuration, and
a sling attached to the third structure.

2. The device of claim 1, wherein the first structure is shaped as a loop.

3. The device of claim 2, wherein the first structure is shaped so that the loop lays in a plane.

4. The device of claim 1, wherein the second structure comprises at least one adjustable bend sufficiently malleable to adjust the support member for proper support in a user's mouth.

5. The device of claim 4, wherein the second structure comprises a pair of adjustable bends.

6. The device of claim 1, wherein the third structure comprises a pair of rods terminating in an upwardly curved V-shape.

7. The device of claim 6, wherein the sling structure forms a concave shape in the V-shaped configuration.

8. The device of claim 1, wherein the third structure follows a contour of the roof of a user's mouth.

9. The device of claim 1, wherein the curved configuration is shaped to straddle an end of the user's soft pallet.

10. The device of claim 1, wherein the sling is shaped to support a user's uvula.

11. The device of claim 1, wherein the sling is shaped to limit movement of a user's uvula.

12. The device of claim 1, wherein the sling is made of a flexible material.

13. The device of claim 1, wherein the support member is made of at least one of: a rod, a tube, and a wire.

14. An intra-oral device comprising a support member coupled to a dental splint, the support member comprising:
a first pair of structures extending outward from the dental split,
a second pair of structure extending outward from the first pair of structures and upward toward a roof of a user's mouth, the second pair of structures sufficiently malleable to adjust the support member in a user's mouth,
a third pair of structures extending from the second pair of structures toward a rear of the user's mouth, the third second pair of structures terminating in an upwardly curved configuration to form a V-shape, and
a concave sling attached to the third structure.

15. The device of claim 14, wherein the first pair of structures extend outward from a loop.

16. The device of claim 15, wherein the loop is shaped so that the loop lays in a plane.

17. The device of claim 6, wherein the sling structure forms a concave shape within the V-shaped configuration.

18. The device of claim 14, wherein the third pair of structures follow a contour of the roof of a user's mouth.

19. The device of claim 14, wherein the curved configuration is shaped to straddle an end of the user's soft pallet.

20. The device of claim 14, wherein the sling is shaped to support a user's uvula.
